(19) United States
(12) Patent Application Publication
(10) Pub. No.: US 2025/0241336 A1
(43) Pub. Date: Jul. 31, 2025

(54) SYSTEMS AND METHODS FOR PROTEIN RECOVERY FROM CELL CULTURE MEDIA

(71) Applicant: Omeat Inc., Los Angeles, CA (US)

(72) Inventors: Alireza Khademhosseini, Los Angeles, CA (US); Seyedvahid Hosseini, Newbury Park, CA (US)

(73) Assignee: Omeat Inc., Los Angeles, CA (US)

(21) Appl. No.: 18/855,752

(22) PCT Filed: Apr. 14, 2023
(86) PCT No.: PCT/US2023/018628

§ 371 Date: Oct. 10, 2024

Related U.S. Application Data
(60) Provisional application No. 63/330,814, filed on Apr. 14, 2022.
(60) Provisional application No. 63/337,554, filed on May 2, 2022.

(51) Int. Cl. A23J 1/00; A23J 3/20; A23J 3/22; A23J 3/26; A23J 3/28; A23L 13/00; C12N 5/077

(52) U.S. Cl. CPC A23J 1/008; A23J 3/20; A23J 3/227; A23J 3/26; A23J 3/28; A23L 13/00; C12N 5/0658; C12N 2500/60; C12N 2531/00

(57) ABSTRACT

The present disclosure generally relates to clean meat and other cultivated animal-derived products. In certain aspects, proteins are harvested from cell culture media (e.g., a protein suspension) being used to culture cells, such as non-human animal cells, to yield a protein isolate. Such cells may produce or secrete proteins, or other biological materials, that are solubilized, or insoluble but in suspension (e.g., exosomes or microvesicles), within the cell culture media, and such proteins, and other biological materials, can be recovered in certain cases (e.g., a protein isolate) and used to produce cultivated meat or other animal-derived products. Examples of soluble proteins in the protein suspension that can be removed from cell culture media include albumin. plasma proteins, other secreted proteins, or the like. Other aspects are generally directed to systems and methods for making or using such proteins, kits involving these, or the like.

### RELATED APPLICATIONS

This application claims the benefit of U.S. Provisional Patent Application Ser. No. 63/330,814, filed Apr. 14, 2022, entitled “Systems and Methods for Protein Recovery from Cell Culture Media,” and of U.S. Provisional Patent Application Ser. No. 63/337,554, filed May 2, 2022, entitled “Systems and Methods of Preparing Cultivated Meat and Meat Analogs from Plant-Based Proteins.” Each of these is incorporated herein by reference in its entirety.

### FIELD

The present disclosure generally relates to cultivated meat and other cultivated animal-derived products.

### BACKGROUND

Non-human cellular agriculture is a promising technology for producing animal-based proteins to solve problems associated with farming live animals. However, high costs are associated with cell culture techniques for cellular agriculture, including expensive cell culture media and bioreactors. These may hinder production of products using cellular agriculture at commercial levels.

In cellular agriculture, after cells proliferate and differentiate in bioreactors, the cells are harvested and assembled into products. Cells may be grown in suspension in cell culture media. One of most important factors to get higher yields of biomass is to increase cell density, e.g., to millions of cells per ml. However, increasing cell density can be challenging, as higher cell densities result in higher consumption of nutrients and production of wastes. Accordingly, such bioreactors are often in need of constant media exchange or recycling. In addition, increases in media usage may be economically unfavorable. Thus, improvements in cell agriculture and cell culture techniques are needed.

### SUMMARY

The present disclosure generally relates to clean meat and other cultivated animal-derived products. The subject matter of the present disclosure involves, in some cases, interrelated products, alternative solutions to a particular problem, and/or a plurality of different uses of one or more systems and/or articles.

One aspect is generally directed to an article. In one set of embodiments, the article comprises a cultivated non-human animal-derived product comprising at least 1 wt % soluble proteins. In another set of embodiments, the article comprises a cultivated non-human animal-derived product comprising at least 0.1 wt % soluble plasma proteins. In yet another set of embodiments, the article comprises a cultivated non-human animal-derived product, comprising cells and microcarriers comprising an animal-derived soluble protein.

The article, in another set of embodiments, comprises microcarriers comprising an animal-derived soluble protein. According to yet another set of embodiments, the article comprises microcarriers comprising an animal-derived plasma protein.

In another set of embodiments, the article comprises a cultivated non-human animal-derived product, comprising aligned myotubes on microcarriers comprising an animal-derived soluble protein. The article, in yet another set of embodiments, comprises a cultivated animal-derived product, comprising an anisotropic scaffold comprising an animal-derived soluble protein. In still another set of embodiments, the article comprises cells cultivated on microcarriers comprising an animal-derived soluble protein.

In other embodiments, the article comprises a cultivated non-human animal-derived product comprising a protein isolate, wherein at least 50 wt % of the protein isolate in the product is matrix proteins. The article, according to one set of embodiments, comprising a cultivated non-human animal-derived product comprising protein isolate, wherein no more than 25 wt % of the protein isolate in the product is plasma proteins. In another set of embodiments, the article comprises a cultivated non-human animal-derived product comprising protein isolate, wherein at least 80 wt % of the protein isolate in the product is denatured. In other embodiments, the article comprises a cultivated non-human-derived product comprising protein isolate, wherein at least 25 wt % of the protein isolate in the product is insoluble. In other embodiments, still, the article comprises a cultivated non-human animal-derived product comprising protein isolate, wherein no more than 25 wt % of the protein isolate in the product is soluble.

Other embodiments relate to articles comprising a cultivated non-human animal derived product comprising protein isolate, wherein at least 50 wt % of the protein isolate in the product is secreted and/or produced in muscle cells. Additionally, some embodiments relate to articles comprising a cultivated non-human animal derived product comprising protein isolate, wherein at least 50 wt % of the protein isolate in the product is secreted and/or produced in liver cells. In other embodiments, still, the articles comprise a cultivated non-human animal derived product comprising protein isolate, wherein at least 50 wt % of the protein isolate in the product is secreted and/or produced by one or more cells. Other embodiments are also contemplated herein. For example, in a one set of embodiments, the article comprises a cultivated non-human animal derived product comprising protein isolate, wherein at least 50 wt % of the protein isolate in the product is harvested from spend cell cultivation media. Another aspect is generally directed to a method. In one set of embodiments, the method comprises removing soluble proteins from cell culture media exposed to non-human animal cells, and producing a cultivated animal-derived product comprising the soluble proteins. The method, in another set of embodiments, comprises removing at least 5 g of protein per liter from cell culture media exposed to non-human animal cells, and producing a cultivated animal-derived product comprising the protein.

In still another set of embodiments, the method comprises culturing non-human animal cells in cell culture media, removing albumin from the cell culture media, and producing a cultivated animal-derived product comprising the albumin and the non-human animal cells. In yet another set of embodiments, the method comprises removing soluble proteins from cell culture media exposed to non-human animal cells, and producing microcarriers comprising the soluble proteins removed from the cell culture media.

The method, in one set of embodiments, comprises seeding myoblasts on microcarriers comprising an animal-derived soluble protein, and causing the myoblasts to fuse to form aligned myotubes on the microcarriers. In another set of embodiments, the method comprises seeding myoblasts on microcarriers comprising an animal-derived soluble protein, growing the myoblasts in a bioreactor to produce aligned myotubes on the microcarriers, and producing a cultivated animal-derived product comprising the aligned myotubes within the bioreactor.

In yet another set of embodiments, the method comprises fabricating microcarriers comprising an animal-derived soluble protein and a plurality of grooves, and seeding myoblasts on the microcarriers. According to still another set of embodiments, the method comprises seeding myoblasts on microcarriers comprising an animal-derived soluble protein and a plurality of grooves, and growing the myoblasts to produce myotubes aligned with the plurality of grooves of the microcarriers.

The method, in another set of embodiments, comprises seeding myoblasts on an anisotropic scaffold comprising an animal-derived soluble protein, and growing the myoblasts to produce a tissue mass comprising the anisotropic scaffold. In yet another set of embodiments, the method comprises removing soluble proteins from cell culture media exposed to non-human animal cells, and extruding the soluble proteins to produce a cultivated non-human animal-derived product comprising the soluble proteins.

In still another set of embodiments, the method comprises removing soluble proteins from cell culture media exposed to non-human animal cells, forming fibers from the soluble protein, and producing a cultivated animal-derived product comprising the fibers. The method, in another set of embodiments, comprises removing soluble proteins from cell culture media exposed to non-human animal cells, ding the soluble proteins to a second cell culture media, and growing cells within the second cell culture media.

Other methods are also contemplated herein, in other sets of embodiments. For example, in a first set of embodiments, the methods comprise culturing microcarriers comprising cells in a cell culture media in a bioreactor, isolating a protein isolate from the cell culture media from the bioreactor, wherein the cell culture media comprises proteins released by the cells, and producing a cultivated animal-derived product comprising the protein isolate. In a second set of embodiments, the methods comprise culturing non-human animal cells in a cell culture media in a bioreactor, wherein the cells release one or more proteins into the cell culture media, isolating at least 5 g of protein per liter of cell culture media exposed to the non-human animal cells, and producing a cultivated animal-derived product comprising the protein.

In a third set of embodiments, the method comprises culturing non-human animal cells in a cell culture media in a bioreactor, wherein the cells release one or more proteins into the cell culture media, removing the proteins from the cell culture media exposed to the non-human animal cells, and producing microcarriers comprising the proteins removed from the cell culture media. In a fourth set of embodiments, the method comprises isolating proteins from a cell culture media following cultivation of non-human animal cells in said media, producing a microcarriers comprising the proteins in the cell culture media and seeding said microcarriers with myoblasts, and causing the myoblasts to fuse to form aligned myotubes on the microcarriers. In a fifth set of embodiments, the methods comprise isolating proteins from a cell culture media following cultivation of non-human animal cells in said media, producing microcarriers comprising the proteins in the cell culture media and seeding said microcarriers with myoblasts, growing the myoblasts in a bioreactor to produce aligned myotubes on the microcarriers, and producing a cultivated animal-derived product comprising the aligned myotubes within the bioreactor.

In a sixth set of embodiments, the methods comprise fabricating microcarriers comprising protein and a plurality of grooves, wherein the protein is isolated from cell culture media following cultivation of non-human animal cells in said media, and seeding myoblasts on the microcarriers. Other embodiments relate to methods comprising seeding myoblasts on microcarriers comprising a protein and a plurality of grooves, wherein the protein is isolated from cell culture media following cultivation of non-human animal cells in said media and growing the myoblasts to produce myotubes aligned with the plurality of grooves of the microcarriers. Additional embodiments relate to methods comprising seeding myoblasts on an anisotropic scaffold comprising a protein, wherein the protein is isolated from cell culture media following cultivation of non-human animal cells in said media, and growing the myoblasts to produce a tissue mass comprising the anisotropic scaffold.

Other methods comprise removing proteins from a cell culture media following cultivation of non-human animal cells in said media and extruding the proteins to produce a cultivated non-human animal-derived product comprising the proteins, according to some embodiments. Additional sets of embodiments relate to methods comprising removing proteins from a cell culture media following cultivation of non-human animal cells in said media, forming fibers from the protein, and producing a cultivated animal-derived product comprising the fibers. Other embodiments still relate to methods comprising removing proteins from a cell culture media following cultivation of non-human animal cells in said media, adding the proteins to a second cell culture media, and growing non-human cells within the second cell culture media.

In another aspect, the present disclosure encompasses methods of making one or more of the embodiments described herein, for example, cultivated non-human animal-derived products. In still another aspect, the present disclosure encompasses methods of using one or more of the embodiments described herein, for example, cultivated non-human animal-derived products.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 is a schematic illustrating the production of proteins by a cell;

FIG. 2 illustrates protein yield versus temperature in accordance with another embodiment;

FIG. 3 illustrates the effect of temperature and heating time on compressive modulus, in yet another embodiment; and

FIG. 4 illustrates bovine myoblasts on microcarriers, in accordance with still another embodiment.

### DETAILED DESCRIPTION

The present disclosure generally relates to clean meat and other cultivated animal-derived products. In certain aspects, proteins are harvested from cell culture media (e.g., a protein suspension) being used to culture cells, such as non-human animal cells, to yield a protein isolate. Such cells may produce or secrete proteins, or other biological materials, that are solubilized, or insoluble but in suspension (e.g., exosomes or microvesicles), within the cell culture media, and such proteins, and other biological materials, can be recovered in certain cases (e.g., a protein isolate) and used to produce cultivated meat or other animal-derived products. Examples of soluble proteins in the protein suspension that can be removed from cell culture media include albumin, plasma proteins, other secreted proteins, or the like. Other aspects are generally directed to systems and methods for making or using such proteins, kits involving these, or the like.

The terms “protein,” “soluble protein,” and “protein suspension” may be used in some cases to refer to any protein present within a cell culture media following cultivation of cells for at least 30 minutes. In some embodiments, the terms “protein” and “protein isolate” can refer to a protein precipitate (or protein aggregate) harvested from the aforementioned cell culture media and encompass the terms “proteins.” “soluble protein,” and “protein suspension.” Cells may generally be thought of as protein production factories, capable of producing a variety of different proteins. These proteins may be kept within the cell (e.g., as intracellular or transmembrane proteins), deposited to the extracellular matrix (e.g., extracellular matrix proteins), or released, e.g., to the environment surrounding the cell (see, e.g., FIG. 1). Although cells produce and release significant amounts of extracellular proteins, such proteins are not always assembled or deposited into the extracellular matrix. Such proteins instead remain soluble within the cell culture media supporting the cells, and are often discarded when the media is exchanged. In some embodiments, however, some proteins may form insoluble aggregates that remain suspended in the cell culture media (e.g., a protein suspension). Other types of insoluble secreted proteins are also possible, such as proteins contained with exosomes and microvesicles. Accordingly, in certain aspects, such proteins may be recovered from the cell culture media, and used for various applications, such as for the production of cultivated meat, or other cultivated animal-derived products.

For instance, in certain embodiments, cells are grown within cell culture media in a bioreactor or other in vitro cell culture system, and the cell culture media may contain or be supplemented with certain components that contain proteins that may aid in cell growth, for example, serum proteins (e.g., plasma, albumin, fetal bovine serum, platelet rich plasma, globulins, etc.), matrix proteins (e.g., collagen, elastin, fibronectin, laminin, glycoproteins, etc.), and plant protein isolates (e.g., plant albumins or plant globulins) or growth factors, such as those described herein. In some embodiments, such proteins may be processed to recover proteins from the media. In some embodiments, proteins recovered from spent culture media may form a protein isolate. For example, the media may be reduced and/or the proteins may be separated from the media, e.g., as discussed herein. A variety of different proteins may be produced by the cells and/or may be dissolved within the media, including proteins such as albumin, globulins, fibronectin, fibrinogen, collagens, or the like. The cells may be animal cells, e.g., non-human animal cells such as chicken, cow, pig, sheep, or other animals including those described herein. In some embodiments, the cells may be isolated from any tissue of the animal cell, such as, the heart, skeletal muscle, brain, skin, liver, kidney, spleen, intestine, or the like.

After growing cells within cell culture media, at least some of the cell culture media may be removed from the cells, e.g., in a continuous process or as various batches, and the media may be processed to remove at least some of the proteins from the cell culture media. For instance, certain proteins may be recovered by using precipitation techniques, such as heating, changes in pH, the addition of certain salts, or the like. Other techniques that can be used for removing proteins are described below. In some embodiments, the protein isolate is recovered by rendering the proteins insoluble. The insoluble proteins, or protein isolate, may self-assemble into protein aggregates, according to some embodiments.

In some embodiments, the protein isolate may self-assemble into protein aggregates having a diameter of between 1 microns and 1000 microns. In some embodiments, the protein aggregates have a diameter of between 10 microns and 900 microns, between 50 microns and 800 microns, between 100 microns and 700 microns, between 200 microns and 600 microns, between and 300 microns and 500 microns.

In some embodiments, the protein isolate may self-assemble into protein aggregates having a diameter of greater than or equal to 1 micron, greater than or equal to 10 microns, greater than or equal to 50 microns, greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, and greater than or equal to 1000 microns. In some embodiments, the protein isolate may self-assemble into protein aggregates having a diameter of less than or equal to 1000 microns, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, less than or equal to 100 microns, less than or equal to 50 microns, less than or equal to 10 microns, and less than or equal to 1 micron.

In some embodiments, the protein isolate may self-assemble into protein aggregates having an aspect ratio between 1:10 to 10:1. In some embodiments, the aspect ratio is greater than or equal to 1:10 greater than or equal to 1:9, greater than or equal to 1:8, greater than or equal to 1:7, greater than or equal to 1:6, greater than or equal to 1:5, greater than or equal to 1:4, greater than or equal to 1:3, greater than or equal to 1:2, greater than or equal to 1:1, greater than or equal to 2:1, greater than or equal to 3:1, greater than or equal to 4:1, greater than or equal to 5:1, greater than or equal to 6:1, greater than or equal to 7:1, greater than or equal to 8:1, greater than or equal to 9:1, and greater than or equal to 10:1. In some embodiments, the aspect ratio is less than or equal to 10:1, less than or equal to 9:1, less than or equal to 8:1, less than or equal to 7:1, less than or equal to 6:1, less than or equal to 5:1, less than or equal to 4:1, less than or equal to 3:1, less than or equal to 2:1, less than or equal to 1:1, less than or equal to 1:2, less than or equal to 1:3, less than or equal to 1:4, less than or equal to 1:5, less than or equal to 1:6, less than or equal to 1:7, less than or equal to 1:8, less than or equal to 1:9, and less than or equal to 1:10.

After precipitation, the recovered proteins (e.g., a protein isolate) may be used in a variety of applications. For example, in certain embodiments, the proteins (e.g., as in a protein isolate) may be formed into a cultivated meat product, for example, that is intended to be eaten by humans. In addition, some cases, the proteins may be combined with other components. For instance, the proteins may be combined with the cells (e.g., lysed cells) giving arise to those proteins, and/or with other cells (e.g., other lysed cells to produce a biomass). In addition, such proteins may be used in other cultivated non-human animal-derived products, for example, leather, cultivated fur, or other products including those described herein. In another example, the recovered proteins may be used to create milk or other dairy products.

A variety of different cells may be used in different aspects to produce the proteins. For example, the cells may be human or non-human, e.g., non-human animal cells. Non-limiting examples of animal cells include chicken, cow, pig, sheep, goat, deer, fish, duck, turkey, shrimp, buffalo, elephant, horse, camel, feline, canine, poultry, crab, bison, crocodile, insects, or any other suitable animal cells. Such cells may be intended for human consumption as food in some embodiments. For example, the cells may include cells such as myoblasts, adipose cells, or the like. However, in some cases, other cell types may be used, e.g., ones that are not intended for human consumption, for example, skin cells, organ cells, or the like.

In one set of embodiments, the cells may be grown in cell culture media within a bioreactor or other in vitro cell culture system. A wide variety of bioreactors can be used in various embodiments including, but not limited to, suspension bioreactors, continuous stirred-tank bioreactors, rocker bioreactors, airlift bioreactors, fixed bed bioreactors, bubble column bioreactors, fluidized bed bioreactors, packed bed bioreactors, or the like. Other examples of cell culture systems include flasks, petri dishes, microwell plates, or the like. Those of ordinary skill in the art will be aware of different bioreactors and other systems for culturing cells in vitro.

In addition, a variety of different cell culture media may be used. Any appropriate cell culture media known in the art may be used to grow the cells disclosed herein. Non-limiting exemplary cell culture media that may be purchased from commercial vendors (e.g., Gibco, Sartorius, etc.), or synthesized, include DMEM, RPMI 1640, MEM, DMEM/F12, Ham's F-10 nutrient mixture, Ham's F-12 nutrient mixture, Media 199, Plasma-Lyte, Essential 8, etc.

Those of ordinary skill in the art will be familiar with techniques for growing cells within a bioreactor or other cell culture system. For instance, cells may be grown at body temperature (e.g., about 38.5° C. for cow cells, about 41° C. for chicken cells, about 39-40° C. for pig cells, about 40-42° C. for duck cells, etc.). Appropriate conditions for culturing cells (temperature, pressure, pH, nutrients, gaseous compositions, CO₂ levels, etc.) will also be known by those of ordinary skill in the art. In addition, the cells may be grown within the bioreactor or other cell culture system for any suitable length of time. For example, the cells may be grown for at least 3 days, at least 5 days, at least 1 week, at least 2 weeks, at least 3 weeks, at least 4 weeks, etc.

As discussed, cells growing within cell culture media may secrete proteins into the cell culture media (e.g., to yield a protein suspension). In some cases, some of the proteins that are secreted are soluble, e.g., within the media. Non-limiting examples include such as albumin, globulin, fibrinogen, collagen, immunoglobulins, antibodies, or the like. In some cases, the proteins that are secreted include proteins found in plasma or serum. In other cases, some of the proteins that are secreted are insoluble. For example, in some cases, some proteins can be secreted within exosomes or microvesicles, according to one set of embodiments. In other cases, at least some of the secreted soluble proteins may form insoluble protein aggregates, for example, after prolonged cell culture, that remain in suspension (e.g., free-floating), according to another set of embodiments. Such insoluble aggregates may be collected in certain embodiments and used to prepare any of the articles disclosed herein using standard techniques known to those of ordinary skill in the art.

Soluble proteins such as these may remain dissolved and/or suspended within the cell culture media. However, in certain aspects such as are discussed herein, such proteins may be removed from the cell culture media and/or from the cells that produced the proteins, e.g., for subsequent use, including those described herein. In some cases, for instance, at least some of the cell culture media may be taken from the bioreactor (or other in vitro cell culture system), with or without cells. The cell culture media may be taken in any suitable fashion, for example, in a continuous process or via periodic batches or aliquots from the bioreactor. As a non-limiting example, in some cases, cells are grown within a bioreactor or other in vitro cell culture system, and the cell culture media is periodically exchanged for fresh cell culture media, e.g., as the cells consume nutrients present within the cell culture media. Such spent cell culture media may be processed as discussed herein to remove at least some proteins from it.

It should be understood that the removal of proteins from the cell culture media and/or from the cells that produced the proteins may not be perfect, e.g., some proteins may remain behind in the cell culture media, and/or some cell culture media may remain with the proteins (e.g., a protein isolate) after their removal from the cell culture media. In some cases, at least 50 wt %, at least 60 wt %, at least 70 wt %, at least 80 wt %, at least 85 wt %, at least 90 wt %, at least 95 wt %, or more of the proteins (e.g., a protein isolate) present in cell culture media may be removed using techniques such as those described herein.

In addition, in certain instances, relatively large amounts of protein (e.g., a protein isolate) may be recovered from the cell culture media. For example, in some cases, at least 5 g, at least 10 g, at least 25 g, at least 50 g, at least 75 g, at least 100 g, at least 125 g, at least 150 g, at least 200 g, or more of protein (e.g., a protein isolate) may be recovered per liter of cell culture media. This is an additional source of protein that has not previously been considered as a suitable source of protein for applications involving cultivated meat, or other cultivated animal-derived products.

A variety of techniques may be used to remove proteins from the cell culture media and/or from the cells that produced the proteins (e.g., to yield a protein isolate), according to certain embodiments. For instance, certain proteins may be recovered by using precipitation techniques to cause at least some of the protein to precipitate, such as heating (e.g., to temperatures of at least 30° C., at least 40° C., at least 50° C., at least 60° C., at least 70° C., at least 80° C., at least 90° C., at least 100° C., at least 110° C., etc.), changes in pH (e.g., acidification using HCl, citric acid, phosphoric acid, H₂SO₄, HNO₃, etc. to pH's of less than 6, less than 5, less than 4.5, less than 4, less than 3.5, less than 3, less than 2.5, less than 2, etc.), the addition of certain salts (e.g., ammonium sulfate, or salts of any of NH₄⁺, K⁺, Na⁺, Li⁺, Mg²⁺, Ca²⁺ and any of F, SO₄²⁻, H₂PO₄⁻, H³CCOO⁻, Cl⁻, NO³⁻, Br⁻, ClO₃⁻, I⁻, ClO, e.g., (NH₄)₂SO₄, NH₄F, K₂SO₄, Li₂SO₄, LiI, NaBr, LiF, etc.), isoelectric precipitation, or the like. Other techniques for recovering protein from solution may also be used, such as centrifugation, filtration, ultrafiltration, diafiltration, chromatographic techniques (e.g., size exclusion chromatography, affinity chromatography, hydrophobic interaction chromatography, etc.), or the like. In some cases, more than one precipitation technique may be used, e.g., serially or simultaneously, etc.

The protein may be recovered from the cell culture media (e.g., to yield a protein isolate) using such techniques and used to produce a cultivated non-human animal-derived product, in accordance with certain aspects. The protein (e.g., a protein isolate) may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the product may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein (e.g., a protein isolate) may comprise between 5 wt % and 10 wt %, between 20 wt % and 50 wt %, between 35 wt % and 55 wt %, etc. of the cultivated non-human animal-derived product.

Additionally, in some cases, relatively large quantities of product may be prepared. For example, the protein that is recovered may have a mass of at least 10 g, at least 25 g, at least 50 g, at least 100 g, at least 300 g, at least 1 kg, etc., and may be introduced into various cultivated non-human animal-derived products such as are described herein.

In some embodiments, a cultivated non-human animal-derived product comprises a protein isolate (e.g. protein) comprising matrix proteins. The matrix proteins may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the matrix proteins may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate may comprise between 5 wt % and 10 wt %, between 20 wt % and 50 wt %, between 35 wt % and 55 wt %, etc. of the cultivated non-human animal-derived product. In some embodiments, at least 50 wt % of the protein isolate in the product is matrix proteins.

The matrix proteins may comprise any proteins recognized in the art as matrix proteins. For example, in some embodiments, the matrix protein comprises collagen (e.g., Type I, Type II, Type III, Type IV. Type V. Type VI. Type VII, Type VIII. Type IX, Type X, Type XI, Type XII, Type XIII, and Type XIV). In some embodiments, the matrix proteins comprise elastin. Other matrix proteins are also possible in other embodiments (e.g., fibronectin and laminin).

In some embodiments, the protein isolate comprises proteoglycans. The protein isolate may comprise any art recognized proteoglycan, for example, heparan sulfate, chondroitin sulfate, and keratan sulfate. Other proteoglycans are possible in other embodiments. In some embodiments, the protein isolate comprises non-proteoglycan polysaccharides. The protein isolate may comprise any art recognized non-proteoglycan polysaccharide, for example, hyaluronic acid.

In some embodiments, a cultivated non-human animal-derived product comprises a protein isolate (e.g. protein) comprising plasma proteins. The plasma proteins may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the plasma proteins may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate may comprise between 5 wt % and 10 wt %, between 20 wt % and 50 wt %, between 35 wt % and 55 wt %, etc. of the cultivated non-human animal-derived product. In some embodiments, at least 25 wt % of the protein isolate in the product is plasma proteins.

The plasma proteins may comprise any proteins recognized in the art as plasma proteins, for example, albumins, globulins, fibrinogen, regulatory proteins, and clotting factors. In some embodiments, the plasma proteins comprise prealbumin (transthyretin), alpha 1 antitrypsin, alpha-1-acid-glycoprotein, alpha-2-macroglobulin, gamma globulins, beta-2 microglobulin, haptoglobin, ceruloplasmin, complement component 3, complement component 4, C-reactive protein, lipoproteins (e.g., chylomicrons, VLDL, LDL, HDL), transferrin, prothrombin, and MBL (or MBP).

In some embodiments, a cultivated non-human animal-derived product comprises a protein isolate (e.g. protein) that is denatured. The denatured protein isolate may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the denatured protein isolate may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate may comprise between 5 wt % and 10 wt %, between 20 wt % and 50 wt %, between 35 wt % and 55 wt %, etc. of the cultivated non-human animal-derived product. In some embodiments, at least 80 wt % of the protein isolate in the product is denatured.

In some embodiments, the protein isolate in the product is denatured using a denaturant. Any denaturant known in the art to denature proteins may be used herein. For example, in some embodiments, the denaturant is heat. Without wishing to be bound by any particular theory, it is generally believed that heating proteins to temperatures of at least 30° C., at least 40° C., at least 50° C., at least 60° C., at least 70° C., at least 80° C., at least 90° C., at least 100° C., at least 110° C., etc. causes the molecules to vibrate more rapidly and violently. The accelerated vibration can disrupt the hydrogen bonds, hydrophobic interaction and Van der Waals forces, causing the unfolding of the proteins three-dimensional and two-dimensional structures.

In some embodiments, the denaturant is a chemical. Those skilled in the art will appreciate that the mechanism of denaturation will be dependent on the particular chemical denaturant. As such, any suitable chemical denaturant may be used to denature any of the proteins disclosed herein. For example, in some embodiments, the denaturant may comprises acids (e.g., acetic acid, trichloroacetic acid, sulfosalicylic acid), bases (e.g., sodium bicarbonate), alcohol (e.g., ethanol), crosslinking agents (e.g., formaldehyde and glutaraldehyde), chaotropic agents (e.g., urea, guanidium chloride, lithium perchlorate, and sodium dodecyl sulfate), disulfide bond reducers (e.g., 2-mercaptoethanol, dithiothreitol, and tris(2-carboxyethyl)phosphine), and chemically reactive agents (e.g., hydrogen peroxide, elemental chlorine, hypochlorous acid, bromine, bromine water, iodine, nitric and other oxidizing acids and ozone). Other chemical denaturants are also possible in other embodiments.

In some embodiments, a cultivated non-human animal-derived product comprises a protein isolate (e.g. protein) that is insoluble. The insoluble protein isolate may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the insoluble protein isolate may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate may comprise between 5 wt % and 10 wt %, between 20 wt % and 50 wt %, between 35 wt % and 55 wt %, etc. of the cultivated non-human animal-derived product. In some embodiments, at least 25 wt % of the protein isolate in the product is insoluble.

In some embodiments, denatured protein isolate and/or insoluble protein isolate may self-assemble into protein aggregates having a diameter of between 1 microns and 1000 microns. In some embodiments, the protein aggregates have a diameter of between 10 microns and 900 microns, between 50 microns and 800 microns, between 100 microns and 700 microns, between 200 microns and 600 microns, between and 300 microns and 500 microns.

In some embodiments, the denatured protein isolate and/or insoluble protein isolate may self-assemble into protein aggregates having a diameter of greater than or equal to 1 micron, greater than or equal to 10 microns, greater than or equal to 50 microns, greater than or equal to 100 microns, greater than or equal to 200 microns, greater than or equal to 300 microns, greater than or equal to 400 microns, greater than or equal to 500 microns, greater than or equal to 600 microns, greater than or equal to 700 microns, greater than or equal to 800 microns, greater than or equal to 900 microns, and greater than or equal to 1000 microns. In some embodiments, the denatured protein isolate and/or insoluble protein isolate may self-assemble into protein aggregates having a diameter of less than or equal to 1000 microns, less than or equal to 900 microns, less than or equal to 800 microns, less than or equal to 700 microns, less than or equal to 600 microns, less than or equal to 500 microns, less than or equal to 400 microns, less than or equal to 300 microns, less than or equal to 200 microns, less than or equal to 100 microns, less than or equal to 50 microns, less than or equal to 10 microns, and less than or equal to 1 micron.

In some embodiments, the denatured protein isolate and/or insoluble protein isolate may self-assemble into protein aggregates having an aspect ratio between 1:10 to 10:1. In some embodiments, the aspect ratio is greater than or equal to 1:10 greater than or equal to 1:9, greater than or equal to 1:8, greater than or equal to 1:7, greater than or equal to 1:6, greater than or equal to 1:5, greater than or equal to 1:4, greater than or equal to 1:3, greater than or equal to 1:2, greater than or equal to 1:1, greater than or equal to 2:1, greater than or equal to 3:1, greater than or equal to 4:1, greater than or equal to 5:1, greater than or equal to 6:1, greater than or equal to 7:1, greater than or equal to 8:1, greater than or equal to 9:1, and greater than or equal to 10:1. In some embodiments, the aspect ratio is less than or equal to 10:1, less than or equal to 9:1, less than or equal to 8:1, less than or equal to 7:1, less than or equal to 6:1, less than or equal to 5:1, less than or equal to 4:1, less than or equal to 3:1, less than or equal to 2:1, less than or equal to 1:1, less than or equal to 1:2, less than or equal to 1:3, less than or equal to 1:4, less than or equal to 1:5, less than or equal to 1:6, less than or equal to 1:7, less than or equal to 1:8, less than or equal to 1:9, and less than or equal to 1:10.

In some embodiments, a cultivated non-human animal-derived product comprises a protein isolate (e.g. protein) that is soluble. The soluble protein isolate may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the soluble protein isolate may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate may comprise between 5 wt % and 10 wt %, between 20 wt % and 50 wt %, between 35 wt % and 55 wt %, etc. of the cultivated non-human animal-derived product. In some embodiments, at least 25 wt % of the protein isolate in the product is soluble.

In some embodiments, a cultivated non-human animal-derived product comprises a protein isolate (e.g. protein) that is secreted by and/or produced in muscle cells. The protein isolate secreted and/or produced from muscle cells may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the protein isolate secreted and/or produced from muscle cells may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate secreted and/or produced in muscle cells may comprise between 5 wt % and 10 wt %, between 20 wt % and 50 wt %, between 35 wt % and 55 wt %, etc. of the cultivated non-human animal-derived product. In some embodiments, at least 50 wt % of the protein isolate in the product is secreted and/or produced in muscle cells.

In some embodiments, the protein isolate secreted by and/or produced in muscle cells comprises one or more myokines. It is recognized in the art that the various combinations and concentration of myokines produced and/or release from muscle cells may be altered by changing the various culture conditions (e.g., composition of the cell culture media, temperature, stir rate, etc.). Thus, all combinations and concentrations of myokines known to be produced and/or secreted by muscle cells are herein contemplated. Non-limiting embodiments include exosomes, microvesicles, myostatin, IGF1, IGF2, TGF-beta, IL6, IL15, Ang1, BDNF, VEGF-A, PDEF, LRP4, and CNTFRa.

In some embodiments, a cultivated non-human animal-derived product comprises a protein isolate or a protein that is secreted by and/or produced in liver cells. The protein isolate secreted and/or produced from liver cells may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the protein isolate secreted and/or produced from liver cells may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate secreted and/or produced in liver cells may comprise between 5 wt % and 10 wt %, between 20 wt % and 50 wt %, between 35 wt % and 55 wt %, etc. of the cultivated non-human animal-derived product. In some embodiments, at least 50 wt % of the protein isolate in the product is secreted and/or produced in liver cells.

In some embodiments, the protein isolate secreted by and/or produced in liver cells comprises one or more proteins. It is recognized in the art that the various combinations and concentration of proteins produced and/or release from liver cells may be altered by changing the various culture conditions (e.g., composition of the cell culture media, temperature, stir rate, etc.). Thus, all combinations and concentrations of proteins known to be produced and/or secreted by liver cells are herein contemplated. For example, in some cases, the protein isolate secreted by and/or produced in liver cells comprises one or more plasma proteins, such as, albumin, alpha-fetoprotein, soluble plasma fibronectin, and C-reactive protein. As a second example, according to some embodiments, the protein isolate secreted by and/or produced in liver cells comprises one or more proteins involved in hemostasis and fibrinolysis. Non-limiting embodiments include alpha2-macroglobulin, alpha1-antitrypsin, antithrombin III, Protein S, Protein C, plasminogen, alpha2-antiplasmin, complement components C1-9 and complement component C3. In another set of embodiments, the protein isolate secreted by an/or produced in liver cells may comprise one or more carrier proteins, for example, albumin, ceruloplasmin, transcortin, haptoglobin, hemopexin, IGF binding protein, major urinary proteins, retinol binding protein, sex hormone-binding globulin, thyroxine-binding globulin, transthyretin, transferrin, and vitamin D-binding protein. In other embodiments, still, the protein isolate secreted by and/or produced in liver cells comprises one or more hormones, for example, FGF21, hepcidin, insulin-like growth factor 1, and thrombopoietin. Combinations of any of these are also possible in various embodiments, e.g., the protein isolate secreted and/or produced in liver cells may comprise plasma protein and/or carrier proteins and/or hemostasis and fibrinolysis proteins and/or hormones.

In some embodiments, a cultivated non-human animal-derived product comprises a protein isolate (e.g. protein) that is secreted by and/or produced by one or more cells. The protein isolate secreted and/or produced by one or more cells may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the protein isolate secreted and/or produced by one or more cells may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate secreted and/or produced in one or more cells may comprise between 5 wt % and 10 wt %, between 20 wt % and 50 wt %, between 35 wt % and 55 wt %, etc. of the cultivated non-human animal-derived product. In some embodiments, at least 50 wt % of the protein isolate in the product is secreted and/or produced in one or more cells.

In some embodiments, a cultivated non-human animal-derived product comprises a protein isolate (e.g. protein) harvested from spent cell cultivation media. The protein isolate harvested from spent cell cultivation media may be used to form any amount or concentration of the product, e.g., at least 0.1 wt %, at least 0.2 wt %, at least 0.5 wt %, at least 1 wt %, at least 2 wt %, at least 5 wt %, at least 10 wt %, at least 15 wt %, at least 20 wt %, at least 25 wt %, at least 30 wt %, at least 35 wt %, at least 40 wt %, at least 45 wt %, at least 50 wt %, at least 55 wt %, at least 60 wt %, at least 65 wt %, at least 70 wt %, at least 75 wt %, at least 80 wt %, at least 85 wt %, at least 90 wt %, at least 95 wt %, etc. of the final product. In some cases, the protein isolate harvested from spent cell cultivation media may form no more than 95 wt %, no more than 90 wt %, no more than 85 wt %, no more than 80 wt %, no more than 75 wt %, no more than 70 wt %, no more than 65 wt %, no more than 60 wt %, no more than 55 wt %, no more than 50 wt %, no more than 45 wt %, no more than 40 wt %, no more than 35 wt %, no more than 30 wt %, no more than 25 wt %, no more than 20 wt %, no more than 15 wt %, no more than 10 wt %, no more than 5 wt %, etc. of the final product. Combinations of any of these are also possible in various embodiments, e.g., the protein isolate harvested from spent cell cultivation media may comprise between 5 wt % and 10 wt %, between 20 wt % and 50 wt %, between 35 wt % and 55 wt %, etc. of the cultivated non-human animal-derived product. In some embodiments, at least 50 wt % of the protein isolate in the product is harvested from spent cell cultivation media.

Additionally, in some cases, relatively large quantities of product may be prepared. For example, the protein (e.g., protein isolate) that is recovered may have a mass of at least 10 g, at least 25 g, at least 50 g, at least 100 g, at least 300 g, at least 1 kg, etc., and may be introduced into various cultivated non-human animal-derived products such as are described herein.

In addition, in certain embodiments, the protein (e.g., a protein isolate) may be combined with other components to form a cultivated non-human animal-derived product. For example, the protein (e.g., a protein isolate) may be combined with the cells (e.g., lysed cell biomass) that originally produced those proteins, for example, that may be harvested from the bioreactor, e.g., after producing such proteins (e.g., soluble proteins, protein suspension, etc.). In some cases, the protein (e.g., a protein isolate) may be combined with cells (e.g., lysed cells) from the same organism that gave rise to the cells producing the protein (e.g. soluble proteins, protein suspension). In certain cases, the protein (e.g., a protein isolate) may be combined with other cells (e.g., lysed cells), for example, of the same or different species as the cells that produced the proteins (e.g., soluble proteins, protein suspension, etc.). Combinations of varying cells and/or other components may also be used in certain instances, e.g., to produce the product. In addition, in some embodiments, the protein (e.g., a protein isolate) may be combined with proteins from other sources, for example, a plant protein.

For instance, in one set of embodiments, the proteins (e.g., a protein isolate) may be mixed with other components, and processed using a variety of techniques to produce the final cultivated non-human animal-derived product. For example, the mixture of proteins (e.g., a protein isolate) and other components may be extruded, 3D-printed, injection molded, etc. to form an animal-derived product. As a specific non-limiting example, components such as muscle replicas, fat replicas, cell lysates, etc. may be used to produce a clean or cultivated meat product.

Accordingly, some embodiments are generally directed to cultivated animal-derived products such as clean or cultivated meat. In some cases, the protein (e.g., a protein isolate) may be combined with non-human animal cells to form a cultivated product. As mentioned, these may be the same cells that produced the proteins (e.g., a protein isolate), or different cells, for example, from the same species or a different species. The proteins (e.g., a protein isolate) may be combined with the non-human animal cells, and/or other components, to produce a cultivated meat product. Examples of such components include, muscle replicas, fat replicas, cell lysates, etc.

For example, muscle replicas may comprise non-human muscle cells, which may in some cases be present on microcarriers or scaffolds, such as those described herein. For instance, such microcarriers or scaffolds may be formed from proteins (e.g., a protein isolates) removed from cell culture media, such as albumin, globulins, fibronectin, fibrinogen, collagens, plasma proteins, etc. Other non-limiting examples of muscle replicas may be found in a patent application entitled “Constructs Comprising Fibrin or Other Blood Products for Meat Cultivation and Other Applications,” filed on Nov. 15, 2021, U.S. Pat. Apl. Ser. No. 63/279,617, incorporated herein by reference in its entirety. In addition, in some cases, the microcarriers or scaffolds may have structures, such as grooves, that may allow the cells such as myoblasts to become aligned in a specific direction, although this is not a requirement. Such structures are described in U.S. Ser. No. 63/159,403, filed Mar. 10, 2021, entitled “Constructs for Meat Cultivation and Other Applications,” by Khademhosseini, et al., incorporated herein by reference in its entirety.

Also, certain embodiments are generally directed to a fat replica. In some cases, the fat replica may comprise fat and a hydrogel. The fat may arise from any suitable source, or more than one source in some instances. For example, the fat may arise from animal cells and/or plants. Examples of plant-based fats include vegetable oil, corn oil, and other oils such as those described herein. Animal-based fats may, in some embodiments, be formed from fat (or adipose) cells that are cultivated, for example, on microcarriers, or other suitable scaffolds. The microcarriers or other scaffolds may comprise fibrin and/or other suitable materials such as those discussed below. In some cases, the fat cells may be grown in a reactor, such as those described herein. As yet another example, the fat may include fat that was synthetically prepared. Other non-limiting examples of fat replicas may be found in a patent application entitled “Systems and Methods of Producing Fat Tissue for Cell-Based Meat Products,” filed on Nov. 15, 2021, U.S. Pat. Apl. Ser. No. 63/279,642, incorporated herein by reference in its entirety.

In one set of embodiments, the fat in the fat replica may be present in an emulsion. An emulsion of fat may be prepared, for example, by emulsifying fat with non-human blood plasma. In some cases, the fat may be caused to form a fat emulsion by mixing the fat with non-human blood plasma. Without wishing to be bound by any theory, it is believed that the plasma has components that can emulsify fat to form fat particles such as chylomicrons. For instance, the plasma may include proteins or surfactants that can from such fat particles.

The non-human blood plasma may be treated in some embodiments to form a fat replica. For example, fibrin within the plasma may be caused to clot and/or by causing the fibrin to crosslink, e.g., by exposing it to thrombin, calcium, or other clotting agents such as those described herein. In addition, in some embodiments, a fat replica may comprise a fat emulsion contained within a hydrogel. The hydrogel may be formed from non-human blood plasma, e.g., as discussed, and/or another component. Non-limiting examples of such hydrogels include alginate, gelatin, or others such as those described herein.

Also, certain embodiments are generally directed to a cell lysate. In some cases, the cell lysate may contain heme, which may be used as a colorant in the cultivated meat product. In one embodiment, the heme may be obtained from non-human red blood cells. For example, red blood cells may be lysed, e.g., by exposing the cells to hypoosmotic or distilled water to form a lysate of non-human red blood cells. Red blood cells contain hemoglobin, a structurally similar protein to myoglobin. Hemoglobin also contains a heme moiety. Lysing the red blood cells may release hemoglobin, e.g., into solution. Furthermore, in addition to these, other methods of lysing red blood cells can be used, including those discussed in more detail herein. Other non-limiting examples of lysates of non-human red blood cells may be found in a patent application entitled “Production of Heme for Cell-Based Meat Products,” filed on Nov. 15, 2021, U.S. Pat. Apl. Ser. No. 63/279,644, incorporated herein by reference in its entirety.

Other non-limiting examples of such components that may be used to form clean or cultivated products include but are not limited to, those described in Int. Pat. Apl. Ser. Nos. PCT/US22/19590, PCT/US22/19594, PCT/US22/19601, PCT/US22/19609, PCT/US22/19615, PCT/US22/19618, PCT/US22/19628, and PCT/US22/19631, each of which is incorporated herein by reference.

It will be appreciated that such clean or cultivated meat products are intended to be eaten, for example, by humans, and that such products will often be formed of edible or digestible materials, e.g., materials that can be digested, or degraded to form generally nontoxic materials within the digestive system. For instance, as discussed, the cultivated meat may contain animal-derived cells, such as muscle cells, fat cells, or the like. These may be formed into ground beef or other meat products for human consumption as food.

In general, cultivated meat products are produced using cells taken from an animal, but then the cells are cultured in vitro, e.g., using bioreactors, flasks, petri dishes, microwell plates, or other cell culture systems. Many cell culture systems will be known to those of ordinary skill in the art. This is in stark contrast to traditional techniques of sacrificing animals and harvesting meat or other organs (e.g., skin, internal organs, etc.) for food or other uses. Although the original cells seeded to form the product may have originated or otherwise have originally been derived from a living animal, the bulk of the cells forming the actual product were grown or cultured in an in vitro setting, e.g., within a bioreactor, rather than naturally as part a living animal.

In addition, it should be understood that the invention is not limited to only cultivated meat products. For example, other food products may be produced using proteins such as described herein, e.g., proteins that are harvested from cell culture media (e.g., protein isolates).

For example, in one embodiment, the protein (e.g., a protein isolate) may be used to produce protein shakes, protein supplements, taste-enhancing additives, flavorings, or the like. In some cases, the protein (e.g., a protein isolate) may be used as a bulking agent, e.g., in a food product. As another example, the protein (e.g., a protein isolate) may be used to form a milk substitute. For example, the protein (e.g., a protein isolate) may be dissolved or be emulsified in an aqueous solution to form a protein-enriched liquid. In some cases, this liquid may be used as a milk substitute, for example, in a variety of cultivated dairy products, such as cheese, yogurt, ice cream, and the like. Examples of suitable emulsifiers that may be used include, but are not limited to lecithin, fatty acids, polyglycerol esters (PGE), polysorbates, stearoyl lactylates, propylene glycol esters (PGMS), sucrose esters, polyglycerol polyricinoleate, ammonium phosphatide, mono- and diglycerides, etc. In yet another example, the cultivated product is a texturized protein, e.g., formed as a meat analog or a meat extender. For example, the protein may be present in a texturized vegetable protein or textured soy protein product.

In some cases, products such as those described herein may be cultivated from animal-derived cells, but the product is not necessarily one that is intended to be eaten. For instance, cells from an animal may be cultured to form various organs that can be harvested, such as skin, hair, fur, or the like. Thus, as a non-limiting example, leather, cultivated fur, etc. can be formed by growing cells in culture, for example as discussed herein, without the traditional method of sacrificing animals to harvest their skin or other organs.

As a non-limiting example, in some cases, the cells may be cultured to grow leather or cultivated fur, and may be derived from an appropriate animal type, e.g., mink or raccoon. In yet another embodiment, the cells may be cultured to grow a product that is to be implanted in a subject. For instance, the cells may be derived from a human, and the product may be a muscle or other organ to be implanted in a human. In one set of embodiments, the cells are derived from the subject (e.g., a human subject) that will receive the implant; this may be useful, for example, to avoid an immunological reaction with the implanted product. As another non-limiting example, organs, tissues, etc. of endangered animals can be grown in accordance with certain embodiments, for example, tiger liver, rhinoceros' horn, etc.

In certain embodiments, animals are farmed to provide cells for cell culture, but are not necessarily slaughtered for their meat. For example, cells may be sourced or sustainably harvested from such animals, then cultured as discussed herein. Accordingly, in certain embodiments, sustainable methods are provided of using animals to supply cells for cell culture. Additionally, it should be noted that cultivated non-human animal-derived products produced in such fashion may contain a significant amount of animal-derived cells and proteins.

Proteins (e.g., protein isolates) removed from cell culture media may be used in a variety of applications. As a non-limiting example, in one aspect, a protein removed from cell culture media (e.g., protein isolates) may be used to form microcarriers, or other scaffolds, which may be used in the production of a cultivated meat product, or other cultivated animal-derived products. In some cases, additional materials may be added to the protein (e.g., protein isolates) to form the microcarriers or other scaffolds. For example, in some cases, additional edible materials may be added, such as fibrin, gelatin, alginate, poly(ethylene glycol), or the like.

In some embodiments, for example, cells may be seeded onto the microcarriers or scaffolds, and grown in vitro, e.g., in a bioreactor or other cell culture systems such as are described herein, to produce a cultivated product. In some cases, the product thus formed can be used without additional processing. As a non-limiting example, a cultivated meat product may be grown by seeding myoblasts on microcarriers or scaffolds, then grown within a bioreactor to form the cultivated meat product. The cultivated meat product may not require subsequent separation or processing steps to convert the cultured cells into a product ready to be cooked or otherwise used as meat. However, it should be understood that in other embodiments, additional steps may be used to convert the cells grown within the bioreactor into a clean meat product, or other cultivated animal-derived product. As mentioned, the cells seeded on the microcarriers, or other types of scaffolds may arise or be derived from any suitable animal.

Accordingly, in one set of embodiments, cells are seeded onto scaffolds. A scaffold may define a substrate that the cells are able to divide and proliferate on, e.g., forming tissue that forms the basis of the cultivated animal-derived product. A variety of cell scaffold structures can be used, including scaffolds known by those of ordinary skill in the art. The scaffold may thus have any suitable size or shape. In some cases, the scaffold may be anisotropic, e.g., not exhibiting radial or spherical symmetry. In addition, in certain embodiments, the scaffold may be relatively solid, or have holes or pores. Thus, for example, the scaffold may have any suitable degree of porosity. One or more than one scaffold may be present. For instance, in certain cases, the scaffold comprises a plurality of microcarriers, e.g., as described herein. If more than one scaffold is present, the scaffolds may be independently the same or different. In addition, in certain embodiments, the scaffold may have one or more grooves, for example, as discussed herein.

In one set of embodiments, a scaffold or microcarrier may have a largest or maximum internal dimension of less than 100 mm, less than 80 mm, less than 70 mm, less than 60 mm, less than 50 mm, less than 40 mm, less than 30 mm, less than 20 mm, less than 10 mm, less than 5 mm, less than 3 mm, less than 2 mm, or less than 1 mm. In addition, in some cases, a scaffold or microcarrier may have a maximum internal dimension that is at least 1 mm, at least 2 mm, at least 3 mm, at least 5 mm, at least 10 mm, at least 20 mm, at least 30 mm, at least 40 mm, at least 50 mm, at least 60 mm, at least 70 mm, at least 80 mm, at least 90 mm, at least 100 mm, etc. In addition, in certain cases, combinations of any of these dimensions are also possible. As non-limiting examples, the microcarriers may have a maximum internal dimension of between 10 mm and 30 mm, between 5 mm and 20 mm, between 3 mm and 10 mm, between 50 mm and 70 mm, between 1 mm and 3 mm, etc. In some cases, the maximum internal dimension is the length of longest straight line that can be contained entirely within the microcarrier and/or the interior of the microcarrier (e.g., if the microcarrier defines a hollow sphere).

In one set of embodiments, the scaffold may take the form of one or more microcarriers. The microcarriers may have any shape or size. In some cases, more than one type of microcarrier may be present, e.g., some of which may have various materials, shapes, sizes, etc., such as are described herein. For example, in some embodiments, at least some of the microcarriers may be substantially spherical or exhibit spherical symmetry, although in other embodiments, at least some of the microcarriers may be non-spherically symmetric or may be anisotropic. In addition, in certain cases, at least some of the microcarriers may have a plurality of grooves, e.g., as discussed herein.

In some cases, however, some or all of the microcarriers may not necessarily be spherical. For example, at least some of the microcarriers may have shapes such as cubical, rectangular solid, tetrahedral, octahedral, irregular, etc. In some cases, at least some of the microcarriers have a shape that is substantially planar. For instance, the microcarrier may have a generally rectangular shape where the smallest dimension of the rectangular solid is substantially smaller than either of the other two dimensions, for example, by a factor of at least 3, at least 5, or at least 10, etc.

The microcarriers, or other scaffolds such as described herein, may be formed using any suitable technique. Non-limiting examples include extrusion, electrospinning, 3D-printing, molding, injection molding, or the like. As still other non-limiting examples, cells may be confined on an engineered surface or material having a micro-nano-topography as contact guidance, or by applying mechanical forces generated either by the contractile activity of the cells or by an external strain.

In one set of embodiments, at least some of the microcarriers or other types of scaffolds may have one or more grooves defined therein. Without wishing to be bound by any theory, it is believed that grooves may promote cellular alignment during growth on the microcarriers or other types of scaffolds. Thus, for example, myoblasts seeded within grooves on microcarriers may be induced to grow together to form substantially aligned myotubes, e.g., that are substantially parallel to each other. This can result in muscle fibers can be grown on the microcarriers. As this is a biological system, those of ordinary skill in the art would understand that the myotubes will not necessarily grow to be perfectly parallel to a high degree of mathematical precision. Nonetheless, the myotubes may still be readily identified as having substantially parallel myotubes within the cultivated animal-derived product, for example, as opposed to myotubes grown on spherical particles not containing grooves, where the myotubes are formed randomly from the myoblasts. For instance, the myotubes may exhibit a strong preference to the direction of the grooves, e.g., having an average directionality that varies by less than 20°, less than 15°, less than 10°, or less than 5° relative to the direction of the grooves.

Thus, in some embodiments, the grooves may be positioned or sized within the microcarriers or other scaffolds to allow the myoblasts to be directional or aligned, e.g., to allow them to fuse together to become myotubes. One or more grooves may be present. For instance, a microcarrier or other scaffold may have at least 2, at least 3, at least 4, at least 5, at least 7, at least 10, at least 12, at least 15, at least 20, at least 25, at least 30, at least 40, at least 50, at least 70, or at least 100 or more grooves defined therein. If more than one type of microcarrier or scaffold is present, they may independently have the same or different numbers of grooves. In some cases, the average number of grooves present within the microcarriers may have the ranges described here. The grooves may be positioned in any orientation on the microcarriers. For instance, the grooves may be substantially parallel to each other, e.g., to promote the formation of substantially aligned myotubes.

As another non-limiting example, a protein removed from cell culture media (e.g., a protein isolate) may be used to form fibers. The fibers may be isotropic or anisotropic. A variety of techniques may be used to form fibers from proteins, including electrospinning, wet-spinning, extrusion spinning, direct spinning, dry spinning, gel spinning or semi-melt spinning, or the like. The fibers may also be formed, woven, knitted, etc. to form woven mats, nonwoven fabrics, or other suitable structures.

As yet another non-limiting example, a protein removed from cell culture media (e.g., a protein isolate) may be added to a second cell culture media, which may be used to culture cells. The cell culture media and the second cell culture media may independently be the same or different, and the cells that produced the protein and cells cultured using the second cell culture media may also independently be the same or different.

Other aspects of the disclosure generally relate to methods of producing a cultivated animal-derived product using the protein isolate disclosed herein. In one set of embodiments, the methods comprise culturing non-human animal cells in a cell culture media in a bioreactor. During cultivation the non-human animal cells release one or more proteins into the cell culture media (e.g., creating a protein suspension). In some embodiments, the methods comprise removing the proteins from a cell culture media following cultivation of non-human animal cells in the media and adding the isolated proteins to a second cell culture media comprising non-human cells and growing the cells in the second cell culture media.

The non-human animal cells may be cultured for any suitable duration of time (e.g., for sufficient time to permit accumulation of secreted proteins in the cell culture media). In some embodiments, the cells are cultured for greater than or equal to 30 min, greater than or equal to 1 hr, greater than or equal to 5 hrs, and greater than or equal to 24 hrs. In some embodiments, the cells are cultured for less than or equal to 24 hrs, less than or equal to 5 hrs, less than or equal to 1 hr, and less than or equal to 30 min. In some embodiments, the cells are cultured for between 1 week and 365 weeks, between 10 weeks and 300 weeks, between 20 weeks and 200 weeks, between 30 weeks and 100 week, between 40 weeks and 90 weeks, between 50 weeks and 80 weeks, and between 60 weeks and 70 weeks.

In some embodiments, the methods comprise culturing microcarriers comprising non-human animal cells in a cell culture media in a bioreactor. Any suitable microcarrier (e.g., support cell adhesion and proliferation) may be used to create any of the articles disclosed herein. Those of skill will understand, however, that articles intended for human consumption will require editable microcarriers (e.g., manufactured using protein isolates), such as those described in U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications.” Articles not intended for human consumption may use non-editable microcarriers (e.g., manufactured using polymers or metallic nanoparticles).

In some embodiments, the methods comprise isolating the one or more proteins released into the cell culture media. Any suitable method known in the art maybe used to isolate the proteins, including those disclosed elsewhere herein. For example, in some cases, the proteins may be isolated by inducing precipitation (e.g., heating with temperatures of at least 30° C., at least 40° C., at least 50° C., at least 60° C., at least 70° C., at least 80° C., at least 90° C., at least 100° C., at least 110° C., etc. (changes in pH such as acidification using HCl, citric acid, phosphoric acid, H₂SO₄, HNO₃, etc. to pH's of less than 6, less than 5, less than 4.5, less than 4, less than 3.5, less than 3, less than 2.5, less than 2, etc.). In some embodiments, the proteins may be isolated by concentrating the cell culture media prior to inducing precipitation, for example, using filtration. Other techniques for recovering protein from solution may also be used, such as centrifugation, ultrafiltration, diafiltration, chromatographic techniques (e.g., size exclusion chromatography, affinity chromatography, hydrophobic interaction chromatography, etc.), or the like. In some cases, more than one precipitation technique may be used, e.g., serially or simultaneously, etc.

In some embodiments, the methods comprise isolating between 5 g and 100 g of protein per liter of cell culture media exposed to the non-human animal cells and producing a cultivated animal-derived product comprising the protein. In some embodiments, the methods comprise isolating greater than or equal to 5 g of protein, greater than or equal to 10 g of protein, greater than or equal to 15 g of protein, greater than or equal to 25 g of protein, greater than or equal to 35 g of protein, greater than or equal to 45 g of protein, greater than or equal to 55 g of protein, greater than or equal to 65 g of protein, greater than or equal to 75 g of protein, greater than or equal to 80 g of protein, greater than or equal to 90 g of protein, and greater than or equal to 100 g of protein per liter of cell culture media exposed to the non-human animal cells. In other embodiments, the methods comprise isolating less than or equal to 100 g of protein, less than or equal to 90 g of protein, less than or equal to 80 g of protein, less than or equal to 75 g of protein, less than or equal to 65 g of protein, less than or equal to 55 g of protein, less than or equal to 45 g of protein, less than or equal to 35 g of protein, less than or equal to 25 g of protein, less than or equal to 15 g of protein, less than or equal to 10 g of protein, and less than or equal to 5 g of protein per liter of cell culture media exposed to the non-human animal cells.

In some embodiments, the methods comprise producing microcarriers comprising the protein isolate disclosed herein (e.g., the protein removed from the cell culture media). In some cases, the microcarriers are edible; in other cases, the microcarriers are inedible. In some embodiments, the microcarriers comprise a plurality of grooves. Without wishing to be bound to any particular theory, it is generally known that protein isolates comprising fibrinogen can be used to fabricate edible microcarriers by converting the fibrinogen to form fibrin. Fibrin forms fibrils which subsequently self-assemble into a non-covalent hydrogel. The stiffness of the fibrin hydrogel may be altered by varying the concentration of fibrinogen and/or controlling the degree of crosslinking (e.g., by varying the concentration of trypsin used to cleave the fibrinogen). Protein isolates may contain other cross-linkable materials as well, in other embodiments. For example, in some embodiments, hyaluronic acid may be isolated from liver cells and used to make microcarriers via gelation following addition of a polyelectrolyte.

In some embodiments, microcarriers produced using the protein isolates disclosed herein are subsequently seeded with a non-human animal derived cell and cultured, for example, in a bioreactor. In some embodiments, the non-human animal derived cells are myoblast cells. In certain embodiments, the myoblast cells fuse together during cultivation to form aligned myotubes on the microcarriers. Myotubes are polynucleated and may comprise between 10 and 1000 nuclei, between 20 and 900 nuclei, between 30 and 800 nuclei, between 40 and 700 nuclei, between 50 and 600 nuclei, between 60 and 500 nuclei, between 70 and 400 nuclei, between 80 and 300 nuclei, and between 90 and 200 nuclei.

In some cases, the microcarriers, produced using the protein isolates disclosed herein, comprising a plurality of grooves. In some cases, the microcarriers are seeded with non-human animal derived cells and cultured, for example, in a bioreactor. The myoblasts, in one set of embodiments, may grow within the plurality of grooves to produce myotubes aligned with the plurality of grooves of the microcarrier. In some embodiments, the myotubes may comprise between 10 and 1000 nuclei, between 20 and 900 nuclei, between 30 and 800 nuclei, between 40 and 700 nuclei, between 50 and 600 nuclei, between 60 and 500 nuclei, between 70 and 400 nuclei, between 80 and 300 nuclei, and between 90 and 200 nuclei.

In some cases, the methods involve fabricating anisotropic scaffolds using the protein isolates disclosed herein. The term “anisotropic” as used herein refers to the quality of exhibiting properties with different values when measured along axes in different directions. In some embodiments, the methods comprise seeding one or more cell types on the anisotropic scaffold and growing the myoblasts to produce a tissue mass comprising the anisotropic scaffold. In some embodiments, the methods comprise growing greater than or equal to 5 g of tissue mass, greater than or equal to 10 g of tissue mass, greater than or equal to 15 g of tissue mass, greater than or equal to 25 g of tissue mass, greater than or equal to 35 g of tissue mass, greater than or equal to 45 g of tissue mass, greater than or equal to 55 g of tissue mass, greater than or equal to 65 g of tissue mass, greater than or equal to 75 g of tissue mass, greater than or equal to 80 g of tissue mass, greater than or equal to 90 g of tissue mass, and greater than or equal to 100 g of tissue mass. In other embodiments, the methods comprise growing less than or equal to 100 g tissue mass, less than or equal to 90 g tissue mass, less than or equal to 80 g tissue mass in, less than or equal to 75 g tissue mass, less than or equal to 65 g tissue mass, less than or equal to 55 g tissue mass, less than or equal to 45 g tissue mass, less than or equal to 35 g tissue mass, less than or equal to 25 g tissue mass, less than or equal to 15 g tissue mass, less than or equal to 10 g tissue mass, and less than or equal to 5 g tissue mass.

In certain embodiments, the methods involve extruding the protein isolates disclosed herein to produce a cultivated non-human animal-product comprising said protein isolates. Any suitable extrusion technique may be used (e.g., hot extrusion, cold extrusion, warm extrusion, friction extrusion, micro extrusion, direct extrusion, indirect extrusion, and hydrostatic extrusion). The skilled artisan will understand that the cultivated non-human animal product may comprise other components, for example, proteins not isolated from the cell culture media, biomass obtained from the cells cultured in said culture media, and the like. In some embodiments, the methods comprise extruding a combination of protein isolates and cultured tissue biomass. In some embodiments, extruding the protein isolate (and/or other proteins) comprises forming fibers and producing a cultivated animal-derived product comprising the fibers (e.g., such as ground beef).

The following are each incorporated herein by reference in their entireties: U.S. Provisional Patent Application Ser. No. 63/159,403, filed Mar. 10, 2021, entitled “Constructs for Meat Cultivation and Other Applications”; U.S. Provisional Patent Application Ser. No. 63/279,617, filed Nov. 15, 2021, entitled “Constructs Comprising Fibrin or Other Blood Products for Meat Cultivation and Other Applications”; U.S. Provisional Patent Application Ser. No. 63/279,631, filed Nov. 15, 2021, entitled, “Methods and Systems of Preparing Cultivated Meat from Blood or Cellular Biomass”; U.S. Provisional Patent Application Ser. No. 63/279,642, filed Nov. 15, 2021, entitled, “Systems and Methods of Producing Fat Tissue for Cell-Based Meat Products”; U.S. Provisional Patent Application Ser. No. 63/279,644, filed Nov. 15, 2021, entitled “Production of Heme for Cell-Based Meat Products”; US Provisional Patent Application Ser. No. U.S. 63/300,577, filed Jan. 18, 2022, entitled “Animal-Derived Antimicrobial Systems and Methods”; U.S. Provisional Patent Application Ser. No. 63/164,397, filed Mar. 22, 2021, entitled “Growth Factor for Laboratory Grown Meat”; U.S. Provisional Patent Application Ser. No. 63/164,387, filed Mar. 22, 2021, entitled, “Methods of Producing Animal Derived Products”; U.S. Provisional Patent Application Ser. No. 63/314,171, filed Feb. 25, 2022, entitled “Growth Factors for Laboratory Grown Meat and Other Applications”; and U.S. Provisional Patent Application Ser. No. 63/314,191, filed Feb. 25, 2022, entitled “Methods and Systems of Producing Products Such as Animal Derived Products.”

In addition, U.S. Provisional Patent Application Ser. No. U.S. 63/330,814, filed Apr. 14, 2022, entitled “Systems and Methods for Protein Recovery from Cell Culture Media,” and U.S. Provisional Patent Application Ser. No. U.S. 63/337,554, filed May 2, 2022, entitled “Systems and Methods of Preparing Cultivated Meat and Meat Analogs from Plant-Based Proteins” are each incorporated herein by reference in its entirety.

The following examples are intended to illustrate certain embodiments of the present disclosure, but do not exemplify the full scope of the disclosure.

### Example 1

This example illustrates the production yield of a bioreactor used for growing bovine myoblasts for cell-extracellular matrix and soluble proteins after cell culture.

A 1-liter glass-stirred bioreactor was prepared by combining cell culture media, fibrin microcarrier (10% v/v) and 100,000 myoblasts/ml. The cell culture was continued for seven days in which the cell density increases to 2 million cells/ml, and resulted in 50 grams of biomass per liter. During cellular proliferation of the bovine myoblasts, the culture media exchanged twice, accounting for a total of 3 liters of cell culture media. The spent culture media was collected, and the protein content of media was precipitated as follows.

The pH of media was decreased to 3.7 below the isoelectric pH of proteins using hydrochloric acid. The media was heated to 100° C., then cooled down to room temperature. During the heating process, it was observed that the clear media solution turned slurry, which appeared to be caused be protein precipitation.

Thereafter, the pH was returned neutral using sodium hydroxide. The media was then centrifuged to remove protein sediment from most of the cell culture media. It was found that each liter of media produced approximately 75 g/L of mass. It should be noted that this was surprisingly more than was collected as cellular biomass of the bovine myoblasts in the bioreactor. In particular, 3 L of used cell culture media produced 225 g of protein mass, as compared to 50 g mass of bovine myoblasts. Thus, in total, 275 g/L of protein was produced from the bioreactor.

Control experiments were also performed to confirm that other soluble components of the cell culture media such as amino acids, vitamins, minerals, etc. were not also precipitated, or did not constitute a significant amount of the recovered precipitant. In particular, cell culture media was processed as described above, but without exposing it to any cells. No turbidity or precipitate was observed, suggesting that only proteins were precipitated, as expected.

### Example 2

This example illustrates the effect of temperature in production yield of recovered protein using spent culture media from a bioreactor used for growing bovine myoblasts after cell culture as described in Example 1.

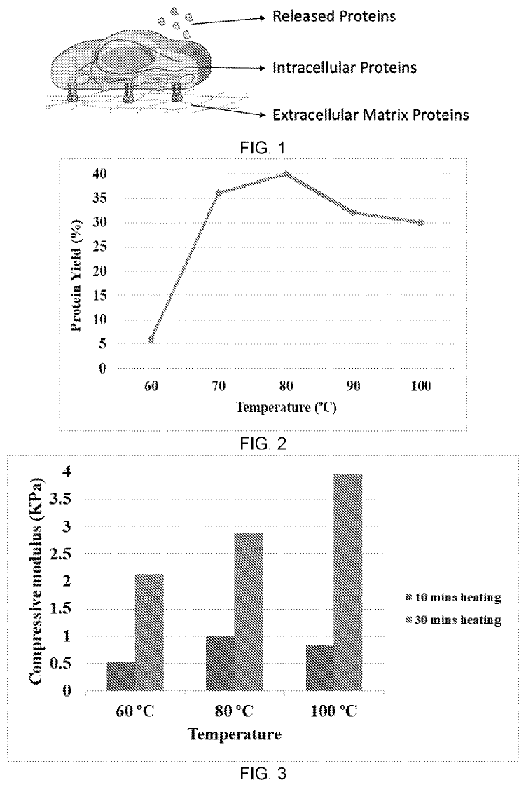

The pH of spent media was decreased to 3.7 below the isoelectric pH of proteins using hydrochloric acid. The media was heated to temperatures of 60° C., 70° C., 80° C., 90° C., 100° C. briefly (flash heat, few second), then cooled down to room temperature. After heat treatment the protein precipitates were separated using a centrifuge at 2000 g for 5 min and the relative volume of precipitates were measured. As illustrated in FIG. 2, the highest yield was achieved at 80° C., while 60° C. had the lowest yield.

### Example 3

This example illustrates a method to produce microcarrier from recovered protein according to prior examples.

In this example, spent cell culture media was used for protein recovery by reduction the pH to 3.7 and flash heating to 80° C. Thereafter, the recovered protein precipitate was collected using centrifugation as described in Example 2. Next, heat treatment was used to cross-link protein particles together. The effect of temperature and heat treatment time was studied on the compressive modulus of the crosslinked protein. Temperatures of 60° C. 80° C., and 100° C., and treatment times of 10 and 30 min were studied. These results showed that highest temperate and heat treatment time increase the compressive modulus according to FIG. 3.

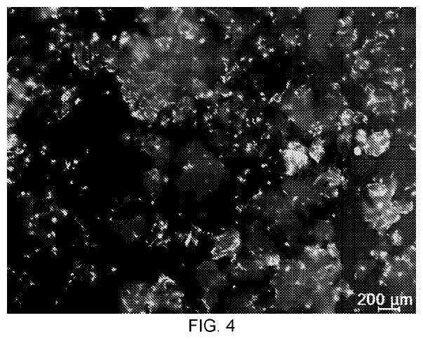

After heat treatment and crosslinking, the protein was broken into small particles using a homogenizer and used as a microcarrier. Bovine myoblasts were seeded on the microcarriers. Proteins treated at 100° C. for 30 min were used for this study. These results showed that the cells were able to bind and proliferate on top of microcarrier particles, as illustrate by Calcein AM staining of living cells and FL microscopy (FIG. 4).

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of.” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a cultivated non-human animal-derived product comprising a protein isolate, wherein at least 50 wt % of the protein isolate in the product is matrix proteins.

2. The article of claim 1, wherein at least 50 wt % of the protein isolate in the product is protein produced by a single cell type.

3. The article of any one of claim 1 or 2, wherein at least 50 wt % of the protein isolate in the product is protein produced by muscle cells.

4. The article of any one of claims 1-3, wherein the matrix proteins comprise collagen.

5. The article of claim 4, wherein the collagen comprises one or more of Type I, Type II, Type III, Type IV, Type V, Type VI, Type VII, Type VIII, Type IX, Type X, Type XI, Type XII, Type XIII, and Type XIV.

6. The article of any one of claims 1-5, wherein the matrix proteins comprise elastin.

7. The article of any one of claims 1-6, wherein the matrix protein comprises fibronectin.

8. The article of any one of claims 1-7, wherein the matrix protein comprises laminin.

9. The article of any one of claims 1-8, wherein the protein isolate further comprises proteoglycans.

10. The article of claim 9, wherein the proteoglycans comprise one or more of heparan sulfate, chondroitin sulfate, and keratan sulfate.

11. The article of any one of claims 1-10, wherein the protein isolate further comprises non-proteoglycan polysaccharides.

12. The article of claim 11, wherein the non-proteoglycan polysaccharide is hyaluronic acid.

13. The article of any one of claims 1-12, wherein the protein isolate further comprises plasma proteins.

14. The article of any one of claims 1-13, wherein the cultivated non-human animal-derived product further comprises a biomass component comprising lysed non-human animal cells.

15. An article, comprising:
a cultivated non-human animal-derived product comprising protein isolate, wherein no more than 25 wt % of the protein isolate in the product is plasma proteins.

16. The article of claim 15, wherein the plasma proteins comprise albumins.

17. The article of any one of claim 15 or 16, wherein the plasma proteins comprise globulins.

18. The article of any one of claims 15-17, wherein the plasma proteins comprise fibrinogen.

19. The article of any one of claims 15-18, wherein the plasma proteins comprise regulatory proteins.

20. The article of any one of claims 15-19, wherein the plasma proteins comprise clotting factors.

21. The article of any one of claims 15-20, wherein the plasma protein comprises one or more of prealbumin (transthyretin), alpha 1 antitrypsin, alpha-1-acid-glycoprotein, alpha-2-macroglobulin, gamma globulins, beta-2 microglobulin, haptoglobin, ceruloplasmin, complement component 3, complement component 4, C-reactive protein, lipoproteins (e.g., chylomicrons, VLDL, LDL, HDL), transferrin, prothrombin, and MBL (or MBP).

22. An article, comprising:
a cultivated non-human animal-derived product comprising protein isolate, wherein at least 80 wt % of the protein isolate in the product is denatured.

23. The article of claim 22, wherein a denaturant is used to denature the protein isolate.

24. The article of claim 23, wherein the denaturant is heat.

25. The article of any one of claim 23 or 24, wherein the denaturant is a chemical.

26. The article of claim 25, wherein the chemical denatures the protein isolate by altering the pH of a solution containing the protein isolate.

27. The article of any one of claims 24-26, wherein the denaturant comprises one or more of acids (e.g., acetic acid, trichloroacetic acid, sulfosalicylic acid), bases (e.g., sodium bicarbonate), alcohol (e.g., ethanol), crosslinking agents (e.g., formaldehyde and glutaraldehyde), chaotropic agents (e.g., urea, guanidium chloride, lithium perchlorate, and sodium dodecyl sulfate), disulfide bond reducers (e.g., 2-mercaptoethanol, dithiothreitol, and tris(2-carboxyethyl)phosphine), and chemically reactive agents (e.g., hydrogen peroxide, elemental chlorine, hypochlorous acid, bromine, bromine water, iodine, nitric and other oxidizing acids and ozone).

28. The article of any one of claims 22-27, wherein the denatured protein isolate lacks a three-dimensional structure.

29. The article of any one of claims 22-28, wherein the denatured protein isolate lacks a two-dimensional structure.

30. An article, comprising:
a cultivated non-human animal-derived product comprising protein isolate, wherein at least 25 wt % of the protein isolate in the product is insoluble.

31. The article of claim 30, wherein the insoluble protein isolate self-assembles into protein aggregates.

32. The article of claim 31, wherein the protein aggregates are between 1 micron and 1000 microns in diameter.

33. The article of claim 31, wherein the protein aggregates are between 50 microns and 500 microns in diameter.

34. The article of any one of claims 31-33, wherein the protein aggregates have an aspect ratio of between 1:10 and 10:1.

35. An article, comprising:
a cultivated non-human animal-derived product comprising protein isolate, wherein no more than 25 wt % of the protein isolate in the product is soluble.

36. An article, comprising:
a cultivated non-human animal-derived product comprising protein isolate, wherein at least 50 wt % of the protein isolate in the product is secreted by and/or produced in muscle cells.

37. The article of claim 36, wherein the protein isolate secreted by and/or produced in muscle cells comprises one or more myokines.

38. The article of claim 37, wherein the myokine comprises one or more of myostatin, IGF1, IGF2, TGF-beta, IL6, IL15, Ang1, BDNF, VEGF-A, PDEF, LRP4, and CNTFRa.

39. The article of any one of claims 36-38, wherein the protein isolate secreted by and/or produced in muscle cells comprises exosomes.

40. The article of any one of claims 36-39, wherein the protein isolate secreted by and/or produced in muscle cells comprises microvesicles.

41. An article, comprising:
a cultivated non-human animal-derived product comprising protein isolate, wherein at least 50 wt % of the protein isolate in the product is secreted by and/or produced in liver cells.

42. The article of claim 41, wherein the protein isolate secreted by and/or produced in liver cells comprises one or more plasma proteins.

43. The article of claim 42, wherein the one or more plasma proteins comprises albumin.

44. The article of any one of claim 42 or 43, wherein the one or more plasma proteins comprises alpha-fetoprotein.

45. The article of any one of claims 42-44, wherein the one or more plasma proteins comprises soluble plasma fibronectin.

46. The article of any one of claims 42-45, wherein the one or more plasma proteins comprises C-reactive protein.

47. The article of any one of claims 42-46, wherein the protein isolate secreted by and/or produced in liver cells comprises one or more factors in hemostasis and fibrinolysis comprises one or more of alpha2-macroglobulin, alpha1-antitrypsin, antithrombin III, Protein S, Protein C, plasminogen, alpha2-antiplasmin, complement components C1-9 and complement component C3.

48. The article of any one of claims 42-47, wherein the protein isolate secreted by and/or produced in liver cells comprises one or more carrier proteins comprises one or more of albumin, ceruloplasmin, transcortin, haptoglobin, hemopexin, IGF binding protein, major urinary proteins, retinol binding protein, sex hormone-binding globulin, thyroxine-binding globulin, transthyretin, transferrin, and vitamin D-binding protein.

49. The article of any one of claims 42-48, wherein the protein isolate secreted by and/or produced in liver cells comprises one or more of FGF21, hepcidin, insulin-like growth factor 1, and thrombopoietin.

50. An article, comprising:
a cultivated non-human animal-derived product comprising protein isolate and cells, wherein at least 50 wt % of the protein isolate in the product is secreted and/or produced by one or more cells.

51. An article, comprising:
a cultivated non-human animal-derived product comprising protein isolate, wherein at least 50 wt % of the protein isolate in the product is harvested from spent cell cultivation media.

52. A method, comprising:
culturing microcarriers comprising cells in a cell culture media in a bioreactor;
isolating a protein isolate from the cell culture media from the bioreactor, wherein the cell culture media comprises proteins released by the cells; and
producing a cultivated animal-derived product comprising the protein isolate.

53. The method of claim 52, wherein isolating the protein isolate from the cell culture media is performed using filtration.

54. The method of any one of claim 52 or 53, wherein isolating the protein isolate from the cell culture media is performed using centrifugation.

55. The method of any one of claims 52-54, comprising isolating at least 5 g of proteins per liter of cell culture media.

56. The method of any one of claims 52-55, comprising isolating at least 75 g of proteins per liter of cell culture media.

57. The method of any one of claims 52-56, wherein isolating the protein isolate comprises exposing the cell culture media to a temperature of at least 60° C.

58. The method of claim 57, wherein exposing the cell culture media to a temperature of at least 60° C. denatures the soluble protein.

59. The method of claim 58, wherein denaturing the protein causes the protein to self-assemble into insoluble protein aggregates.

60. The method of any one of claims 52-59, wherein isolating the protein isolate comprises exposing the cell culture media to a pH of less than 4.

61. The method of claim 52-60, wherein isolating the protein isolate comprises exposing the cell culture media to a pH of less than 4 and a temperature of at least 60° C.

62. The method of any one of claims 52-61, wherein isolating the protein isolate comprises exposing the cell culture media to phosphoric acid.

63. The method of any one of claims 52-62, wherein isolating the protein isolate comprises exposing the cell culture media to HCl.

64. The method of any one of claims 52-63, wherein isolating the protein isolate comprises exposing the cell culture media to citric acid.

65. The method of any one of claims 52-64, wherein exposing the cell culture media to a pH of less than 4 causes the protein isolate to denature and to self-assemble into insoluble protein aggregates.

66. A method, comprising:
culturing non-human animal cells in a cell culture media in a bioreactor, wherein the cells release one or more proteins into the cell culture media;
isolating at least 5 g of protein per liter of cell culture media exposed to the non-human animal cells; and
producing a cultivated animal-derived product comprising the protein.

67. The method of claim 66, comprising isolating at least 75 g of protein per liter from the cell culture media.

68. A method, comprising:
culturing non-human animal cells in a cell culture media in a bioreactor, wherein the cells release one or more proteins into the cell culture media;
removing the proteins from the cell culture media exposed to the non-human animal cells; and
producing microcarriers comprising the proteins removed from the cell culture media.

69. A method, comprising:
isolating proteins from a cell culture media following cultivation of non-human animal cells in said media;
producing a microcarriers comprising the proteins in the cell culture media and seeding said microcarriers with myoblasts; and
causing the myoblasts to fuse to form aligned myotubes on the microcarriers.

70. The method of claim 69, further comprising producing the protein by culturing non-human animal cells in cell culture media, and separating the animal-derived protein from the cell culture media.

71. A method, comprising:
isolating proteins from a cell culture media following cultivation of non-human animal cells in said media;
producing microcarriers comprising the proteins in the cell culture media and seeding said microcarriers with myoblasts;
growing the myoblasts in a bioreactor to produce aligned myotubes on the microcarriers; and
producing a cultivated animal-derived product comprising the aligned myotubes within the bioreactor.

72. The method of claim 71, further comprising producing the protein by culturing non-human animal cells in cell culture media, and separating the animal-derived protein from the cell culture media.

73. A method, comprising:
fabricating microcarriers comprising protein and a plurality of grooves, wherein the protein is isolated from cell culture media following cultivation of non-human animal cells in said media; and
seeding myoblasts on the microcarriers.

74. The method of claim 73, further comprising producing the protein by culturing non-human animal cells in cell culture media, and separating the animal-derived protein from the cell culture media.

75. A method, comprising:
seeding myoblasts on microcarriers comprising a protein and a plurality of grooves, wherein the protein is isolated from cell culture media following cultivation of non-human animal cells in said media; and
growing the myoblasts to produce myotubes aligned with the plurality of grooves of the microcarriers.

76. The method of claim 75, further comprising producing the protein by culturing non-human animal cells in cell culture media, and separating the animal-derived protein from the cell culture media.

77. A method, comprising:
seeding myoblasts on an anisotropic scaffold comprising a protein, wherein the protein is isolated from cell culture media following cultivation of non-human animal cells in said media; and
growing the myoblasts to produce a tissue mass comprising the anisotropic scaffold.

78. The method of claim 77, further comprising producing the protein by culturing non-human animal cells in cell culture media, and separating the animal-derived protein from the cell culture media.

79. A method, comprising:
removing proteins from a cell culture media following cultivation of non-human animal cells in said media; and
extruding the proteins to produce a cultivated non-human animal-derived product comprising the proteins.

80. The method of claim 79, wherein the cultivated non-human animal-derived product is a clean meat product.

81. The method of any one of claim 79 or 80, wherein the cultivated non-human animal-derived product is a ground beef replica.

82. The method of any one of claims 79-81, further comprising extruding the proteins and a muscle replica to produce a cultivated non-human animal-derived product.

83. The method of any one of claims 79-82, further comprising extruding the proteins and a fat replica to produce a cultivated non-human animal-derived product.

84. The method of any one of claims 79-83, further comprising extruding the proteins and a cell lysate to produce a cultivated non-human animal-derived product.

85. The method of any one of claims 82-84, comprising extruding the soluble proteins and a red blood cell lysate to produce a cultivated non-human animal-derived product.

86. A method, comprising:
removing proteins from a cell culture media following cultivation of non-human animal cells in said media;
forming fibers from the protein; and
producing a cultivated animal-derived product comprising the fibers.

87. The method of claim 86, wherein at least some of the fibers are anisotropic.

88. The method of any one of claim 86 or 87, wherein at least some of the fibers are isotropic.

89. The method of any one of claims 86-88, wherein forming the fibers from the protein comprises electrospinning the fibers from the protein.

90. The method of any one of claims 86-89, wherein forming the fibers from the soluble protein comprises wet-spinning the fibers from the soluble protein.

91. The method of any one of claims 86-90, comprising forming the fibers into a mat.

92. A method, comprising:
removing proteins from a cell culture media following cultivation of non-human animal cells in said media;
adding the proteins to a second cell culture media; and
growing non-human cells within the second cell culture media.

93. An article, comprising:
a cultivated non-human animal-derived product comprising at least 1 wt % soluble proteins.

94. An article, comprising:
a cultivated non-human animal-derived product comprising at least 0.1 wt % soluble plasma proteins.

95. A method, comprising:
removing soluble proteins from cell culture media exposed to non-human animal cells; and
producing a cultivated animal-derived product comprising the soluble proteins.

96. A method, comprising:
removing at least 5 g of protein per liter from cell culture media exposed to non-human animal cells; and
producing a cultivated animal-derived product comprising the protein.

97. A method, comprising:
culturing non-human animal cells in cell culture media;
removing albumin from the cell culture media; and
producing a cultivated animal-derived product comprising the albumin and the non-human animal cells.

98. An article, comprising:
a cultivated non-human animal-derived product, comprising cells and microcarriers comprising an animal-derived soluble protein.

99. An article, comprising:
microcarriers comprising an animal-derived soluble protein.

100. An article, comprising:
microcarriers comprising an animal-derived plasma protein.

101. A method, comprising:
removing soluble proteins from cell culture media exposed to non-human animal cells; and
producing microcarriers comprising the soluble proteins removed from the cell culture media.

102. An article, comprising:
a cultivated non-human animal-derived product, comprising aligned myotubes on microcarriers comprising an animal-derived soluble protein.

103. The article of claim 66, wherein the animal-derived soluble protein is produced by cultured cells.

104. An article, comprising:
a cultivated animal-derived product, comprising an anisotropic scaffold comprising an animal-derived soluble protein.

105. An article, comprising:
cells cultivated on microcarriers comprising an animal-derived soluble protein.

106. A method, comprising:
seeding myoblasts on microcarriers comprising an animal-derived soluble protein; and
causing the myoblasts to fuse to form aligned myotubes on the microcarriers.

107. A method, comprising:
seeding myoblasts on microcarriers comprising an animal-derived soluble protein;
growing the myoblasts in a bioreactor to produce aligned myotubes on the microcarriers; and
producing a cultivated animal-derived product comprising the aligned myotubes within the bioreactor.

108. A method, comprising:
fabricating microcarriers comprising an animal-derived soluble protein and a plurality of grooves; and
seeding myoblasts on the microcarriers.

109. A method, comprising:
seeding myoblasts on microcarriers comprising an animal-derived soluble protein and a plurality of grooves; and
growing the myoblasts to produce myotubes aligned with the plurality of grooves of the microcarriers.

110. A method, comprising:
seeding myoblasts on an anisotropic scaffold comprising an animal-derived soluble protein; and
growing the myoblasts to produce a tissue mass comprising the anisotropic scaffold.

111. A method, comprising:
removing soluble proteins from cell culture media exposed to non-human animal cells; and
extruding the soluble proteins to produce a cultivated non-human animal-derived product comprising the soluble proteins.

112. A method, comprising:
removing soluble proteins from cell culture media exposed to non-human animal cells;
forming fibers from the soluble protein; and
producing a cultivated animal-derived product comprising the fibers.

113. A method, comprising:
removing soluble proteins from cell culture media exposed to non-human animal cells;
adding the soluble proteins to a second cell culture media; and
growing cells within the second cell culture media.
